(19) United States
(12) Patent Application Publication
(10) Pub. No.: US 2022/0015620 A1
(43) Pub. Date: Jan. 20, 2022

(54) ENDOSCOPE AND METHOD OF USE

(71) Applicant: Meditrina, Inc., Cupertino, CA (US)

(72) Inventors: Csaba Truckai, Saratoga, CA (US); Daniel Truckai, Saratoga, CA (US); Khoi Le, San Jose, CA (US); Kevin Moss, Tracy, CA (US); Britta Nelson, Loomis, CA (US); Nicholas Landgraf, Huntington Woods, MI (US)

(73) Assignee: Meditrina, Inc., Cupertino, CA (US)

(21) Appl. No.: 17/490,643

(22) Filed: Sep. 30, 2021

Related U.S. Application Data
(63) Continuation of application No. 15/836,460, filed on Dec. 8, 2017.
(60) Provisional application No. 62/433,121, filed on Dec. 12, 2016.

(51) Int. Cl. A61B 1/307; A61B 1/05; A61B 1/005; A61B 1/015; A61B 1/06; A61B 1/00; A61B 1/018

(52) U.S. Cl. CPC A61B 1/307; A61B 1/05; A61B 1/005; A61B 1/018; A61B 1/0676; A61B 1/00071; A61B 1/00052; A61B 1/015

(57) ABSTRACT

Endoscopes include a shaft having a diameter extending about a longitudinal axis to a distal housing. An image sensor is carried by the distal housing, and a channel extends through the shaft and distal housing. A distal portion of the channel is adjustable or curved to accommodate introduction of a tool while maintaining a reduced shaft diameter. In some embodiments, a diagonal dimension of the image sensor when combined with a channel diameter is greater than a shaft diameter. In some embodiments, the distal housing is flexible to accommodate passage of a straight tool through a curved distal portion of a working channel. In some embodiments, a curved distal portion of a working channel may be reoriented to deflect a flexible tool.

### CROSS-REFERENCE TO RELATED APPLICATION

This application is a continuation of U.S. patent application Ser. No. 15/836,460 (Attorney Docket No. 50553-705.201), filed Dec. 8, 2017, which claims the benefit of provisional application No. 62/433,121 (Attorney Docket No. 50553-705.101), filed on Dec. 12, 2016, the full disclosure of which is incorporated herein by reference.

### BACKGROUND OF THE INVENTION

### 1. Field of the Invention

The present invention relates to an endoscope assembly, and more particularly to an endoscope with a working channel for use in hysteroscopy, and a method of use of the endoscope assembly.

Endoscopes are used in a wide variety of minimally invasive surgical procedures, including laparoscopy, arthroscopy, and the like. Of particular interest to the present application, hysteroscopy is a minimally invasive procedure for resecting fibroids and performing similar interventions in a patient's uterus. Hysteroscopy utilizes a hysteroscope which is a type of endoscope that carries optics for viewing, a light source for illumination, and a working channel. Interventional tools, such as an electrosurgical loop or other cutter, forceps, and the like, can be introduced though the working channel of the hysteroscope to perform a therapeutic procedure while the patient's uterus is insufflated. The hysteroscope is often introduced through a passage in a transcervical sheath which also allows insufflation of the uterine cavity.

Heretofore, to accommodate the optics, light sources, and the working channel, hysteroscopes have had large diameters which require passage of a large sheath through the cervix, further requiring dilation of the cervix prior to insertion. Cervical dilation requires stretching the cervix with a series of dilators of increasing diameter, and can be traumatic for many patients.

For these reasons, it would be desire able to provide hysteroscopes having relatively small diameters to reduce or eliminate the need to dilate the patient's cervix prior to introduction of the hysteroscope. It would be further desirable to provide methods utilizing such hysteroscopes, and still further desirable to provide similar designs and methods for all types of endoscopes used in a variety of minimally invasive procedures including, laparoscopy, arthroscopy, and the like. At least some of these objectives will be met by the inventions described and claimed hereinafter.

### SUMMARY OF THE INVENTION

The present invention provides an endoscope and a method for using the endoscope in hysteroscopies and other endoscopic surgical procedures. The endoscope design of the present invention provides a low profile which facilitates introduction through small body passages, such as patient's cervix, and into body cavities, such a patient's uterus. Particular endoscope designs incorporate a number of features which can be used alone or in combination to achieve the certain objectives of the present invention, such as a reduced endoscope shaft diameter and reduced patient trauma during introduction of the endoscope.

In a first aspect, an endoscope constructed in accordance with the principles of the present invention comprises a shaft having a diameter and extending along a longitudinal axis. A distal housing is disposed at a distal end of the shaft, and an image sensor is carried by the distal housing, usually on a distal face of the housing which is generally transverse to the longitudinal axis. The imaging sensor is typically rectangular and has a diagonal dimension measured from a first corner to a second diagonally opposed corner. The shaft is usually cylindrical and has a diameter or width and a channel which extends through the shaft to the distal housing, typically from a proximal end of the shaft which is optionally connected to a handle. The has a distal portion channel extends through the distal housing, and at least that distal portion is adjustable in shape to accommodate interventional or other tools as they are introduced through a shaft portion of the channel and into the distal portion of the channel that lies in the distal housing. In accordance with the present invention, a combination or sum of (1) the diagonal dimension and (2) a width or diameter dimension of the channel is greater than a diameter dimension of the shaft. These relative dimensions can maximize the width or diameter of the working channel while minimizing the diameter of the shaft which must be introduced into the patient.

In specific examples of this endoscope, the diagonal dimension of the image sensor alone is greater than fifty percent (50%) of the shaft diameter dimension, often greater than sixty percent (60%) of the shaft diameter dimension, and sometimes greater than seventy percent (70%) of the shaft diameter dimension. In still other specific examples, the channel diameter dimension alone is greater than thirty percent (30%) of the shaft diameter dimension, often greater than forty percent (40%) of the shaft diameter dimension, and sometimes greater than fifty percent (50%) of the shaft diameter dimension. In still further specific examples, the distal housing will be adjustable between a straight shape and a curved shape, typically being deformed from the straight shape to the curved shape when an interventional tool is introduced through the channel. In still other specific examples, the channel portion of the distal housing is further adjustable between and at least a partially collapsed shape and a non-collapsed shape, typically being opened to the non-collapsed shape by introduction of an interventional tool through the working channel. In yet further examples, the distal housing may be formed, at least in part, from an elastomeric material, where the elastomeric material properties allow the distal housing to be adjustable in shape as described previously. In such examples, the remainder of the shaft may be formed from a non-elastomeric material, such as a metal or a hard or rigid plastic. In still further specific examples, the image sensor will be carried in a generally transverse orientation relative to the longitudinal axis and will be inclined at a small angle resulting in a non-orthogonal relative position. In the latter case, the orientation of the image sensor may change as an interventional tool is introduced through the working channel, deforming the distal housing so that the image sensor assumes an orthogonal position relative to the longitudinal axis. In still other specific examples, the distal housing may carry a prism, lens, or other optical element which will alter or adjust the field-of-view of the image sensor to accommodate any non-orthogonal positioning or for other reasons.

In a second aspect, an endoscope constructed in accordance with the principles of the present invention comprises a shaft extending along a longitudinal axis to a distal housing. An image sensor is carried on or by the distal housing, typically being oriented generally transversely to the longitudinal axis on a distal end or face of the housing. A working channel extends through the shaft as well as through the distal housing and can accommodate introduction of an interventional tool therethrough. A portion of the housing approximate a distal end of the shaft comprises a shape-adjustable component. The shape-adjustable component permits a portion of the working channel in the distal housing to deflect relative to a portion of the working channel in the shaft as a tool is advanced through the working channel from the shaft into the distal housing.

In specific examples of this second endoscope design, the shape-adjustable component may comprise any one of an elastomeric material, a flexible material, a hinged component, and the like. The materials and the hinged component may be separate components or may be an integral portion of the housing, e.g., the housing may be formed, at least partly, from the elastomeric material or flexible material to accommodate bending. In still further examples, the distal housing may have a straight cylindrical shape to facilitate insertion into the patient's body, for example through a cervix, and will adjust to a non-straight or non-linear shape to accommodate the tool as it is introduced through the working channel. Typically, the tool will be generally straight and will straighten the working channel as it deflects the distal housing away from the longitudinal axis. In still other examples, the distal housing has a repose or “relaxed” position in which the working channel has a non-straight or non-linear shape in a tensioned or deflected position where the working channel is straightened to accommodate tool introduction therethrough, e.g., a straight tool will straighten an initially deformed or deflected distal housing and working channel. In yet further examples, the endoscope shaft may further comprise a flow channel extending through the shaft and/or distal housing to an open termination or port to allow the introduction of fluids and/or aspiration during a procedure. Often, these endoscopes will further have dimensions of the working channel and the image sensor diagonal which, when combined, are greater than a cross-sectional dimension of the shaft. In some instances, the diagonal dimension of the image sensor is greater than fifty percent (50%) of the cross-sectional dimension of the shaft. In other instances, a diameter dimension of the working channel is greater than fifty percent (50%) of a cross-sectional dimension of the shaft.

In a third aspect, an endoscope constructed in accordance with the principles of the present invention comprises an elongated member, such as a shaft, extending along a longitudinal axis through a proximal portion and an elastomeric distal portion. An image sensor is carried on or by the elastomeric portion, and the elastomeric portion is aligned with the longitudinal axis in a “repose” or “relaxed” configuration for introduction into a patient's body, typically through a body passage into a body cavity. The elastomeric portion of the elongate member is configured or adapted to deform to a tensioned or deflective configuration when a tool is introduced through a working channel of the elongated member.

In specific examples of this third endoscope, a central axis of the working channel may be out of alignment with the longitudinal axis of the elongated member, i.e. an axis of the working channel is not parallel to the longitudinal axis of the shaft. For example, a central axis of the working channel in the elastomeric portion of the elongated member may be diverged, inclined, or otherwise deflected away from the longitudinal axis while in the repose position. In other instances, a central axis of the working channel may diverge away from the longitudinal axis in an angled or curved configuration. In still other instances, the elongated member may comprise a flow channel extending there through to an open termination in the elastomeric portion, typical for providing fluids or aspiration. In still other instances, first and second flow channels may extend through the elongated member to respective first and second open terminations or ports in the elastomeric portion.

In a fourth aspect, an endoscope constructed in accordance with the principles of the present invention comprises the elongated member extending about a central axis through a proximal portion and a distal portion. An image sensor is mounted on or carried by the distal portion of the elongated member, and a working channel extends through the elongated member. The elongated member typically has a straight configuration for introduction into a patient's body and a deflective configuration for accommodating a tool introduced through the working channel.

In specific examples of this fourth endoscope, the distal portion of the elongated member deflects relative to a proximal portion of the elongated member in the deflected configuration for accommodating the tool shaft. In other specific instances, the elongated member may comprise a hinge structure at an interface between the proximal portion and the distal portion of the elongated member, for example being an elastomeric or other “living” hinge. In other instances, the distal portion may comprise in whole or in part an elastomeric body. In still other specific instances, a diagonal dimension of the image sensor will be at least fifty percent (50%) of the diameter of the elongated member in the straight configuration. In other instances, a mean cross-sectional dimension of the working channel is at least thirty percent (30%) of a diameter dimension of the elongated member in the straight configuration. In still other instances, the image senor is carried at a non-orthogonal angle relative to the central axis, for example at a selected angle relative to said central axis which provides a field-of-view that encompasses a working space distal to the working channel. Such an angle may be in the range from 45° to 90° relative to the central axis. In still other instances, a prism or other lens may be positioned adjacent to the image sensor for adjusting or modifying a field-of-view of the image sensor.

In a fifth aspect of the present invention, a method for imaging and treating a body cavity comprises providing an endoscope having any of the features and combinations of features described above, for example including an elongated member extending about a central axis through a proximal portion and a distal portion, an image sensor carried by the distal portion, and a working channel extending through the elongate member. The endoscope is advanced into the body cavity, and the body cavity is imaged using the imaging sensor. A tool may then be advanced through the working channel of the elongate member into the body cavity. The tool and the image sensor diverge relative to each other as the tool is advanced through the distal portion of the elongate body, and the tool may then be used to treat the body cavity while the tool and the image sensor remain diverged relative to each other.

In specific examples of this method, the tool may be advanced through the working channel to cause the distal end of the elongated member to deflect relative to the central axis. Typically, the tool will remain straight as the distal end of the elongated member deflects. Alternatively, a tool may be advanced through the working channel to cause the tool to deflect. In such instances, the working channel is typically reoriented within the elongated member after then the scope has been advanced into the body cavity. Typically, the working channel will comprise a curved sleeve, and reorienting will comprise rotating the curved sleeve about an axis parallel to the central axis. In all instances, the image sensor may be an optical image sensor having a field-of-view projecting in a generally distal direction relative to the distal portion. The field-of-view of the imaging sensor is typically inclined relative to the central axis before the tool and the imaging sensor diverge relative to each other. The field-of-view is then general parallel to the central axis after the tool and the image sensor have diverged relative to each other.

### BRIEF DESCRIPTION OF THE DRAWINGS

Additional aspects of the invention will become clear from the following description of an illustrative embodiment and from the attached drawings, in which:

FIG. 1 is a perspective view of an embodiment of an endoscope corresponding to the invention.

FIG. 2 is a perspective view of a distal portion of the endoscope shaft including a resilient, elastomeric distal portion that carries an image sensor and LEDs, showing the distal portion in a straight insertion configuration.

FIG. 3 is a perspective view sectional view of a portion of the shaft in phantom view, the elastomeric distal portion and the image sensor of FIG. 2 taken along line 3-3 of FIG. 2 in the straight insertion configuration of FIG. 2.

FIG. 4A is a longitudinal sectional view through a portion of the shaft and the distal elastomeric portion of the endoscope in an insertion configuration.

FIG. 4B is another longitudinal sectional view similar to that of FIG. 4A with the distal elastomeric portion in a deformed or displaced configuration after being deflected by a rigid tool shaft inserted through a working channel in the endoscope.

FIG. 5A illustrates a method of use of the endoscope of FIGS. 1-4B in a hysteroscopy wherein a cervical sealing assembly is provided and thereafter the endoscope shaft is introduced through the seal assembly into a patient's uterine cavity in an insertion configuration as shown in FIGS. 1, 2, 3 and 4A further showing the field-of-view of the image sensor.

FIG. 5B illustrates a subsequent step of the hysteroscopy method of FIG. 5A wherein a treatment tool is introduced through the endoscope shaft which deflects the distal elastomeric portion to provide the deployed or displaced configuration.

FIG. 6 is a longitudinal sectional view through a shaft and distal elastomeric portion of an alternative embodiment of an endoscope in an insertion configuration.

FIG. 7 is a sectional view similar to that of FIG. 6 with the distal elastomeric portion in a rotated configuration after being deflected by a rigid tool shaft inserted therethrough.

FIG. 8 is a perspective view of a variation of an endoscope shaft and a non-elastomeric distal portion wherein a working channel exiting a side of the working end.

FIG. 9 is a longitudinal sectional view through the shaft and distal portion of the embodiment of FIG. 8.

FIG. 10 is a cut-away view of the shaft and distal portion of the embodiment of FIGS. 8 and 9.

FIGS. 11A-11B illustrate another variation of endoscope shaft which carries an interior rotatable sleeve with a working channel therein.

FIG. 11C is another view of variation of endoscope shaft of FIGS. 11A-11B with a tool introduced through the working channel.

FIGS. 12A-12B illustrates another variation of endoscope shaft that carries a resilient interior sleeve with a working channel therein.

FIGS. 13A-13B illustrates another variation of endoscope shaft that carries a resilient interior sleeve with a working channel therein.

### DETAILED DESCRIPTION OF THE INVENTION

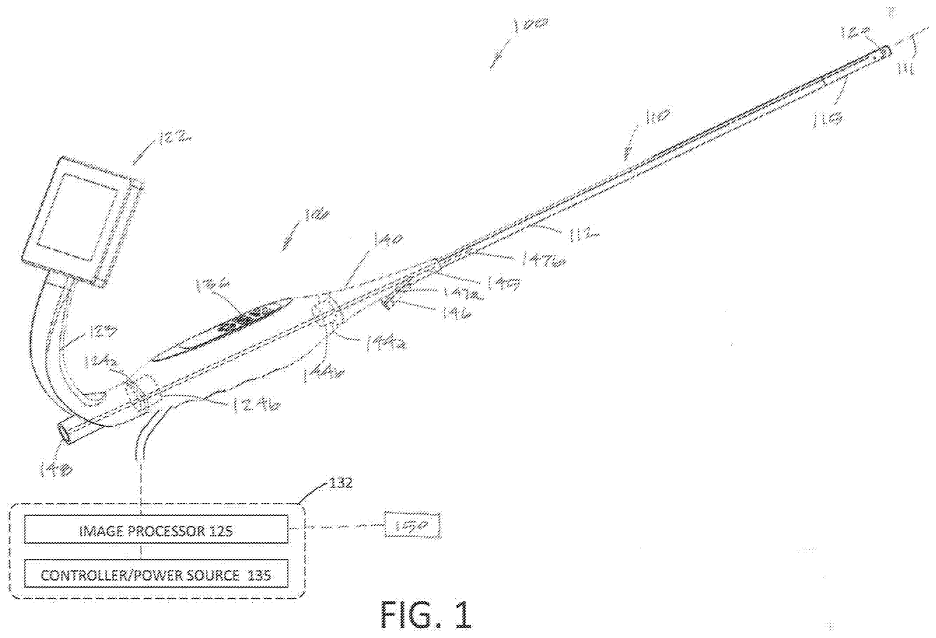

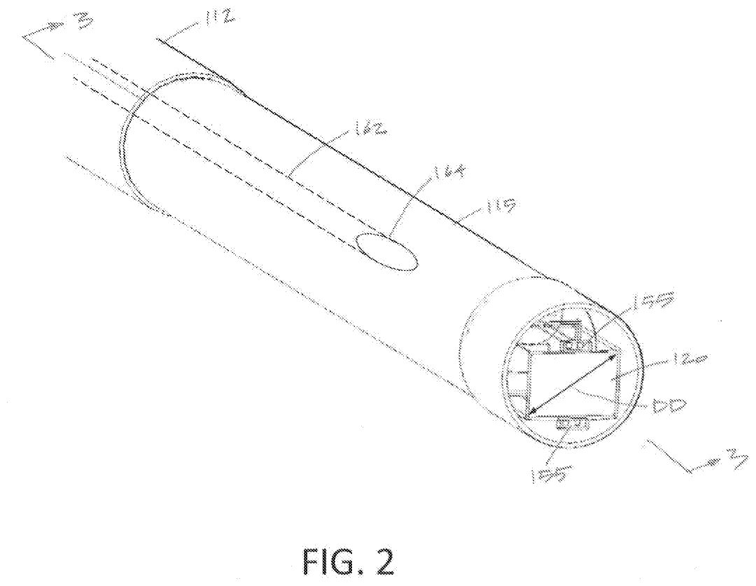

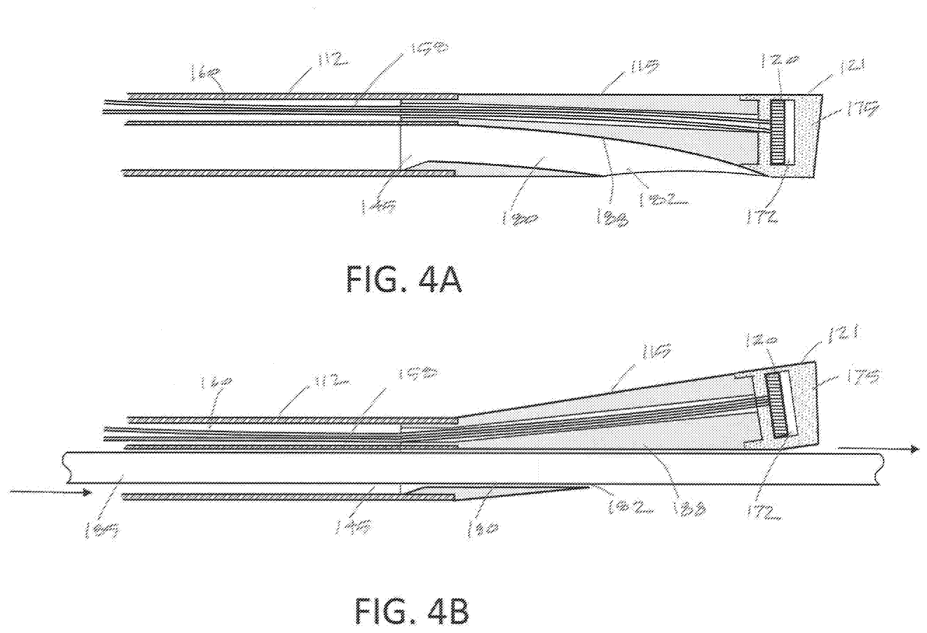

FIG. 1 illustrates an endoscope 100 corresponding to the invention which comprises a proximal handle portion 106 is coupled to a shaft portion 110 extending along longitudinal axis 111. The shaft includes a rigid proximal portion 112 that extends to a flexible, resilient housing or elastomeric distal portion 115. An electronic image sensor 120, typically an optical image sensor, and more typically a CCD or equivalent device, is carried in the elastomeric distal portion 115 of the shaft as shown in FIG. 2. The image sensor 120 is covered by a transparent tip member 121 (not shown in FIG. 2) that can be seen in FIG. 4A. The transparent tip 121 can further comprise a focusing lens and/or a prism for modifying the sensor's field-of-view. In one variation, the handle 106 carries a detachable image display 122 that has coupling member 123 configured with a display connector 124a that mates with handle connector 124b. The image sensor 120 is further operatively connected to an image processor 125 carried in a remote base unit 132 together with a controller/power source 135 for the sensor 120 and LEDs described below. Alternatively, the image processor 125 or components thereof can be carried in the handle 106. A control pad 136 is provided in the handle with actuator buttons for operating the system and image sensor, for example to turn on/off the image sensor 120, capture still images, adjust light from LEDs, etc.

In one variation, the shaft 110 extends distally from a hub 140 that is detachably coupled to handle 106 wherein hub connecter 144a mates with handle connector 144b. In some variations, the shaft 110 may be rotated while the handle 106 is adapted for being held in a stable position. Thus, the handle 106 and display 122 can positioned at a selected angle by the physician, and the shaft 110 can be rotated to orient the image sensor 120 in a selected rotational direction when in use. Such rotation can be accomplished by a rotating grip (not shown) in the hub 140 or in the shaft adjacent the hub 140.

In one variation, shaft 110 has a diameter ranging between 2.5 mm and 10 mm with a length configured for use in hysteroscopy. More commonly, the shaft diameter is from 4 mm to 6 mm in diameter.

As will be described below, the handle 106 and shaft 110 are configured with a working channel 145 that may have a diameter ranging between 1 mm and 6 mm. The working channel or tool-receiving channel 145 is adapted for receiving various types of tools. For example, a biopsy device may have a flexible shaft (not shown) with a diameter ranging from 1 mm to 3 mm and can be introduced through port 146 on the hub 140 which extends through a curved path 147a to a straight channel 147b in the shaft 110. Alternatively, a tissue resecting device (not shown) can be used which may have a larger rigid shaft with a diameter, for example, from 2.5 mm to 5 mm. Such a rigid shaft tool may be introduced through port 148 in display coupling member 123 and handle as shown in FIG. 1. The endoscope or the endoscope shaft 110 may be disposable or re-usable. In one variation, the shaft portion 110 is disposable as described above and is detachable from the handle 106 which is reusable.

As can be seen in one variation in FIG. 1, the display 122 is adapted for detachable coupling to the handle 106. In another system variation, the display 122 does not have to be attached to handle 106 and the image processor 125 controller 135 can send images signals to a remote display 150 (see FIG. 1) such as a monitor in an endoscopic viewing and recording system as is known in the art.

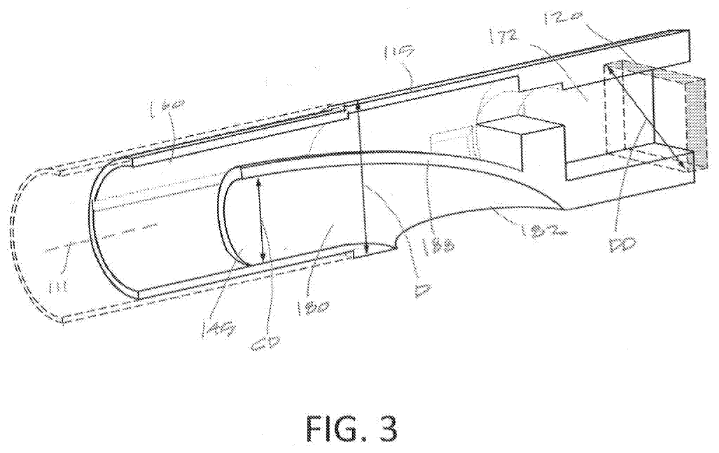

Now turning to FIGS. 2 and 3, the distal elastomeric portion 115 also carries one or more light emitters, for example, LEDs indicated at 155. The image sensor 120 can be coupled to the image processor 125 by wire leads 158 (FIGS. 4A and 4B) which can be independent wires or an elongated flex circuit extending through passageway 160 in the shaft 110 and elastomeric portion 115. Similarly, wire leads (not shown) connect the LEDs to the remote electrical source and controller 140.

Still referring to FIG. 2, it can be seen that a flow channel 162 extends through the shaft 110 and has an open termination 164 in the distal elastomeric portion 115. Such a flow channel 162 can be used for either fluid inflows or fluid outflows from a working space or for measuring pressure in the working space with a static fluid channel. The proximal end of the flow channel 162 can communicate with a Luer fitting in the housing 140 (not shown). It should be appreciated that first and second flow channels with open distal terminations can be provided, with Luer connections in the hub 140 as just described with such channels being used for more the one of the purposes described above. In one variation, a fluid management system can be coupled to inflow and outflow channels in the endoscope to provide a circulating flow through a patient's uterine cavity and can maintain a set intra-cavity pressure as is known in the art.

Now referring to FIG. 3, a longitudinal sectional view of the elastomeric distal portion 115 or housing is shown. The image sensor 120 is carried in a distal region of the housing 115. A transparent distal tip 121 is shown in FIG. 4A that can comprise a clear material such as a plastic lens material which is sealed and coupled to the distal end of the housing 115. The elastomeric portion 115 and transparent tip 121 coupled together to provide a space 172 therein that carries the image sensor 120. In a variation shown in FIGS. 4A-4B, it can be seen that the transparent distal tip 121 further comprises a prism 175 for altering the direction of the field-of-view of the image sensor 120 as will be described below.

Referring again to FIG. 3, the endoscope shaft 110 and elastomeric portion 115 is shown in an insertion profile or configuration wherein the elastomeric distal portion 115 is in a repose, non-tensioned position and the working channel 145 has a distal portion 180 that is curved with an open termination 182 in the side or bottom of the elastomeric portion 115.

FIG. 4A shows the elastomeric portion 115 in another schematic view again in the straight insertion configuration. In FIG. 4B, it can be seen that when the physician inserts a rigid tool shaft 185 through the working channel 145 it will interface with the wall 188 of the repose, curved working channel portion 180 in the elastomeric portion 115. Continued advancement of the tool shaft 185 through the working channel 145 and curved repose channel portion 180 will cause the curved channel portion 180 to straighten until the working end of the tool exits the open termination 182 of the working channel 145. In other words, elastomeric portion 115 is deformed or displaced to a tensioned position wherein the image sensor 120 is moved away from the longitudinal axis of the shaft 110. When the shaft 185 of the tool is withdrawn from the working channel, the elastomeric portion 115 will return from the tensioned position of FIG. 4B to the repose or non-tensioned position of FIG. 4A.

In general, the endoscope corresponding to the invention allows for the use of an image sensor 120 having a large diagonal dimension relative to the insertion profile of the endoscope shaft 110 while at the same time providing a working channel 145 that has a large channel diameter CD relative to the insertion profile of the endoscope shaft 110. More in particular, the endoscope comprises a shaft having a shaft diameter SD extending about a longitudinal axis 111 to a distal housing 115, an image sensor with a diagonal dimension DD carried by the distal housing 115, and a working channel having a diameter CD extending through the shaft and distal housing, wherein the channel portion in the distal housing is adjustable in shape to accommodate a tool introduced therethrough and wherein the combined sensor's diagonal dimension DD and the channel diameter CD is greater than the shaft diameter SD (see FIG. 3). In a variation, the sensor diagonal dimension DD is greater than 50% of the shaft diameter SD, greater than 60% of the shaft diameter or greater than 70% of the shaft diameter. In a variation, the working channel diameter CD is greater than 30% of the shaft diameter, greater than 40% of the shaft diameter or greater than 50% of the shaft diameter. In other words, the working channel portion in the distal housing is adjustable between a curved shape and a straight shape. In another variation described below, the channel portion in the distal housing is adjustable between an at least partially collapsed shape and a non-collapsed shape.

In another aspect of the invention, the image sensor 120 can be carried in a non-orthogonal position relative to the longitudinal axis of the shaft 110 to orient the sensor's field-of-view to be aligned with a working space distal from the end of the endoscope after a tool is inserted through the working channel 145. In a variation, the image sensor 120 can be carried by the elastomeric portion 115 at an angle ranging between 45° to 90° relative to the longitudinal axis 111 of the proximal shaft portion 112 to provide a selected field-of-view.

In another aspect of the invention, the endoscope comprises a shaft extending about a longitudinal axis to a distal housing, an image sensor 120 carried by the distal housing 115 and a working channel 145 extending through the shaft and distal housing wherein a portion of the housing proximate the image sensor and the working channel comprises a shape-adjustable component or wall 188 as shown in FIG. 3. The shape-adjustable component 188 comprises at least one of an elastomeric material, a flexible material and a hinged component. The endoscope shaft 110 and distal housing 115 have a straight cylindrical shape for insertion into a patients' body and is capable of adjustment to a non-straight shape for accommodating a tool introduced through the working channel 145. In a variation, the portion of the working channel 180 in the distal housing 115 is adjustable between a non-straight shape and a straight shape (see FIG. 3). The elastomeric distal housing 115 has a repose position in which the working channel 145 has a non-straight shape and a tensioned position wherein the working channel has straight shape for accommodating a tool introduced therethrough. In a variation, the diagonal of the image sensor is greater than 50% of the cross-section of the shaft and the diameter of the working channel is greater than 50% of the cross-section of the shaft.

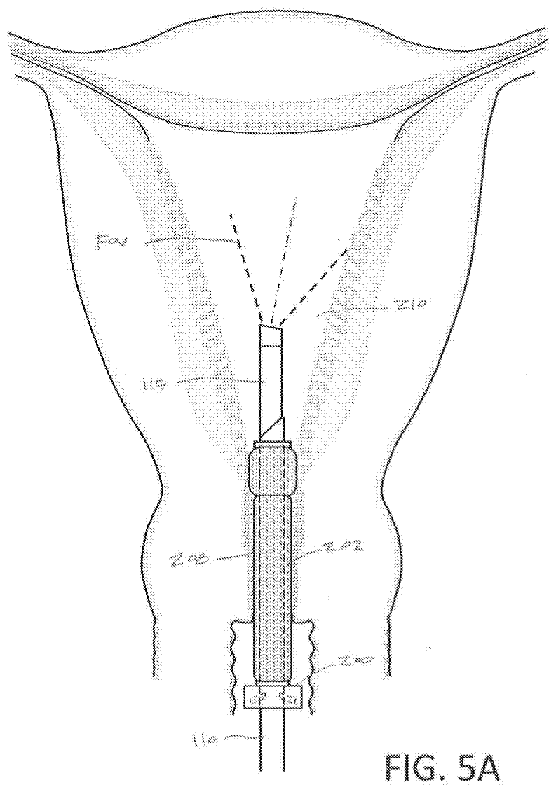

FIG. 5A illustrates a method of the invention to carry out a planned hysteroscopic procedure, wherein an introducer 200 with a cervical seal structure 202 is inserted into the patient's endocervical canal 208 to access the uterine cavity 210. The cervical seal 202, for example, can be a balloon that is expanded to provide an occlusive seal. Other types of cervical seals are known in the art and may be used such as foams, plugs, a seal member with elastomeric fins and the like. After positioning the seal 202 in the endocervical canal 208, the physician then may use a fluid management system adapted for use with inflow and outflow channels (not shown) through the introducer 200 for distending the uterine cavity. A typical fluid management system may provide a circulating flow through the patient's uterine cavity 210 and also to maintain a set fluid pressure therein.

Thereafter, the endoscope 100 and display 122 are assembled (see FIG. 1) and coupled to the controller 135. Next, still referring to FIG. 5A, the endoscope shaft 110 is introduced through the introducer 200 so that the elastomeric distal portion 115 of the endoscope is positioned in the patient's uterine cavity 210. The physician then may examine the patient's uterine cavity and diagnose any abnormalities.

In one example, the physician may identify abnormal tissue in the uterine cavity 210, such as adhesion, polyp or submucosal fibroid. The physician then may elect to treat the abnormal tissue with a suitable tool that can be introduced through the working channel 145 in the endoscope 100. In one example shown in FIG. 5B, the physician elects to use a scissor-like tool 220 for resecting an adhesion or a polyp. The tool 220 has a shaft 222 which may be rigid and has a diameter ranging from 2.5 mm to 5 mm that is configured for mechanical cutting or resection of tissue. As can be seen in FIG. 5B, the introduction of the rigid shaft 222 of the resection tool 220 through the working channel 145 causes deflection of the elastomeric distal portion 115 to thus provide a straight pathway through the endoscope shaft 110 past the deflected elastomeric portion 115 to a working space indicated at 228.

FIG. 5B also illustrates that the field-of-view FOV of the image sensor 120 and prism 175 is oriented so that the working end 240 of the tool 220 and the working space 228 is effectively in the center of such a field-of-view FOV.

In general, an endoscope of the invention comprises an elongated member extending about a longitudinal axis through a proximal portion and a distal elastomeric portion, an image sensor carried by the elastomeric portion wherein the elastomeric portion is aligned with the longitudinal axis in a repose configuration for introduction into a patient's body and wherein the elastomeric portion is adapted for deformation to a tensioned configuration by a tool introduced through a working channel therein. In this variation, the central axis of the working channel in the repose position is not aligned with the longitudinal axis 111 of the shaft 110. The central axis of the working channel in the elastomeric portion in the repose position diverges away from said longitudinal axis 111 in a curve or at an angle.

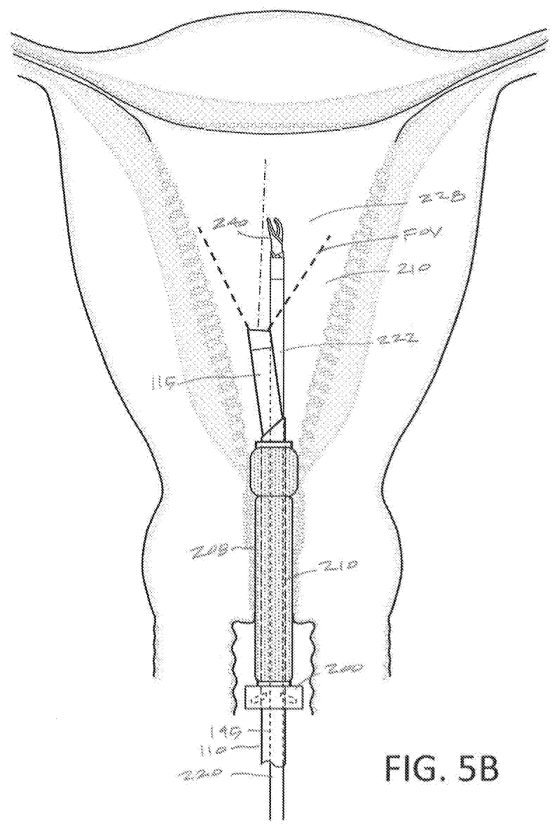

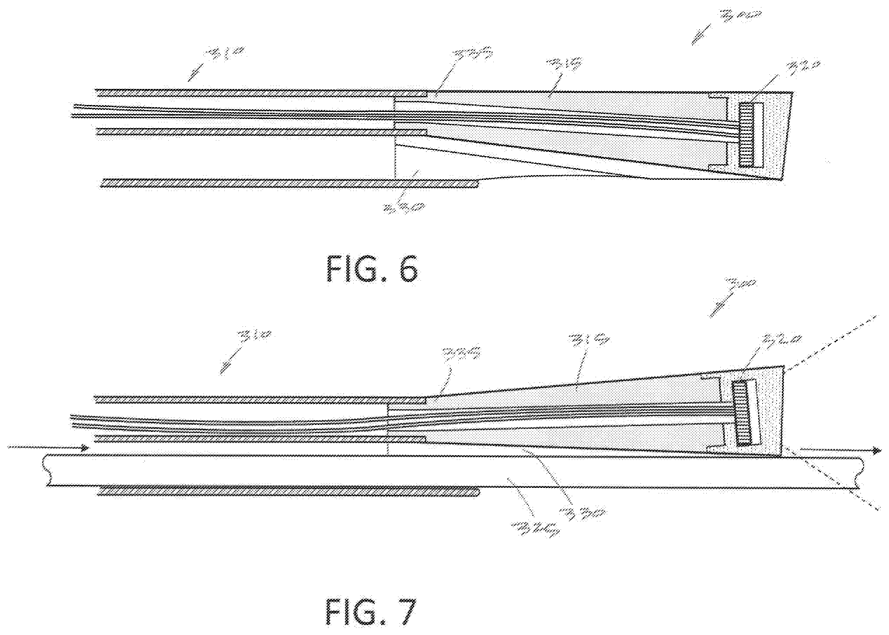

FIGS. 6 and 7 show another endoscope 300 with a distal end that is similar to that described previously. Endoscope shaft 310 extends to a distal elastomeric portion 315 that carries an imaging sensor 320 and LEDs (not shown). FIG. 6 illustrates the elastomeric distal portion 315 in a repose position. FIG. 7 illustrates a tool shaft 325 being introduced through the working channel 330 of elastomeric distal portion 315. In this variation, elastomeric distal portion 315 effectively provides a living hinge indicated at 335 about which the distal portion 315 rotates. In this variation, there is no curved channel through the elastomeric body which is effectively straightened by the tool shaft. It should be appreciated that another similar variation could be made with a non-elastomeric distal portion 315 similar to that of FIG. 6, and a pin-type hinge, that optionally has a spring to return the distal portion to the straight position.

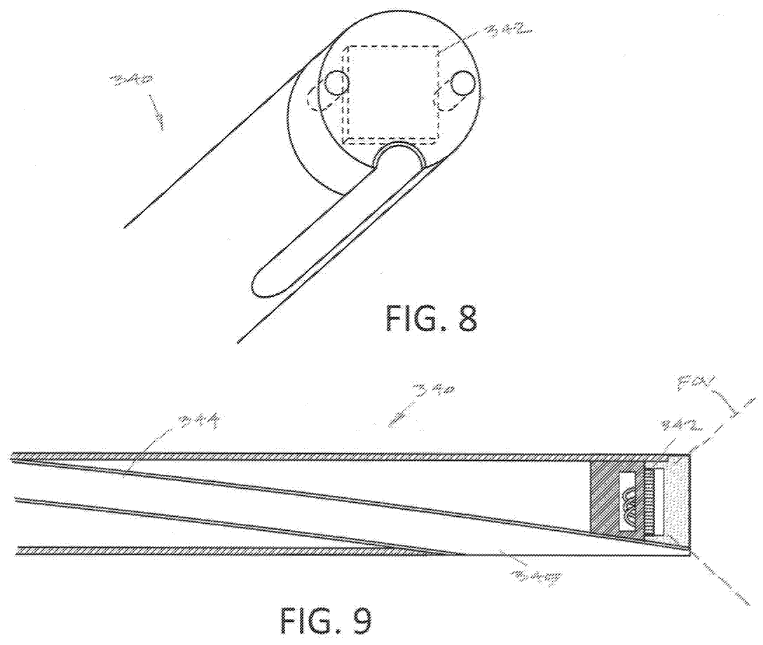

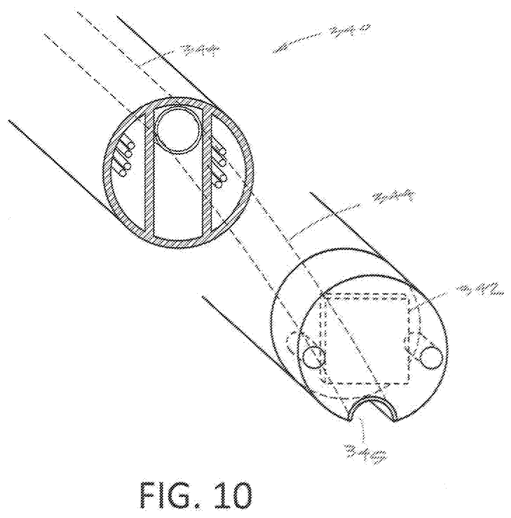

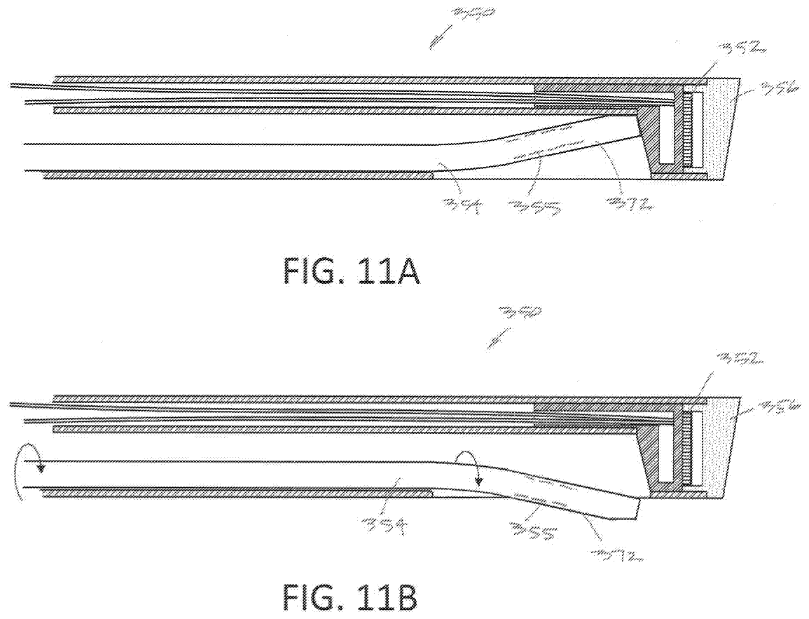

FIGS. 8, 9 and 10 illustrate another variation of an endoscope shaft 340 that does not have an elastomeric distal portion, but still provides an image sensor 342 and a working 344 channel with a combined dimension cross-sectional dimension that is greater than the diameter of shaft 340. In this variation, an elongated curved working channel 344 is provided with the distal open termination 345 on the side of the shaft. This curved working channel 344 is thus adapted only for flexible shaft tools. The image sensor 342 has a field-of-view that is wide enough to observe the working end of the tool as it exits the shaft at a low angle relative to the shaft axis, which angle can be from 5° to 30°.

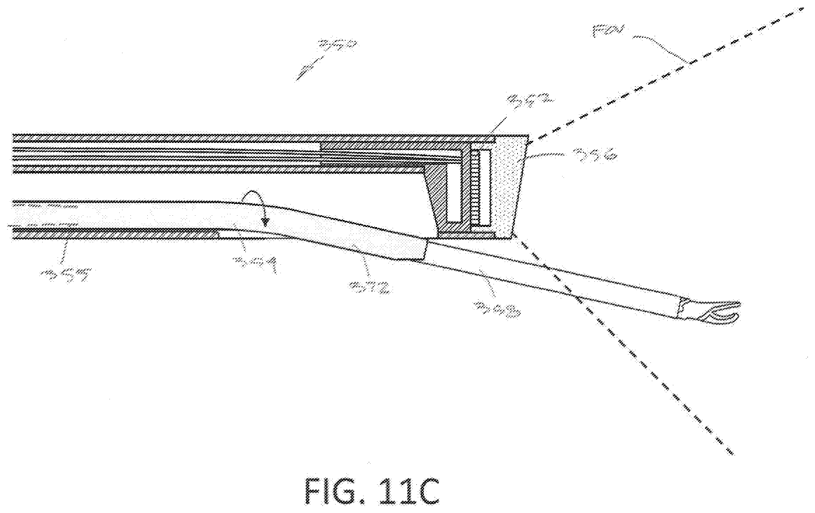

FIGS. 11A-11B illustrate another variation of an endoscope shaft 350 which carries an image sensor 352 and an interior curved sleeve 354 having a working channel 355 therein that can be rotated outwardly from the shaft 350. Again, this variation is adapted for use with the flexible shaft tool 358. Further, a prism 356 is provided proximate the image sensor 352 to adjust the field-of-view FOV to better observe the working end of the tool. In this variation, it can be seen that the interior sleeve 354 is rigid with a curved distal portion 372.

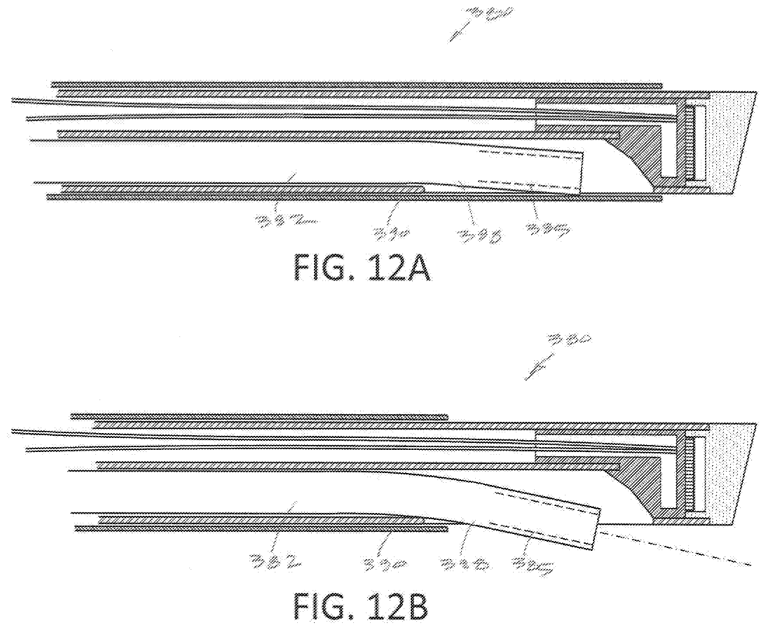

FIGS. 12A and 12B illustrate another variation of endoscope shaft 380 that has an interior sleeve 382 with a working channel 385 therein. The interior sleeve 382 comprises a resilient material with its distal portion 388 a having a repose curved shape which can be constrained by an outer sleeve 390 around the endoscope. As can be seen in FIG. 12B, the outer sleeve 390 can be withdrawn to thus allow the distal portion 388 of interior sleeve 382 to the deflect outwardly and to accommodate a flexible shaft tool and thus function similar to the embodiment shown in FIG. 11.

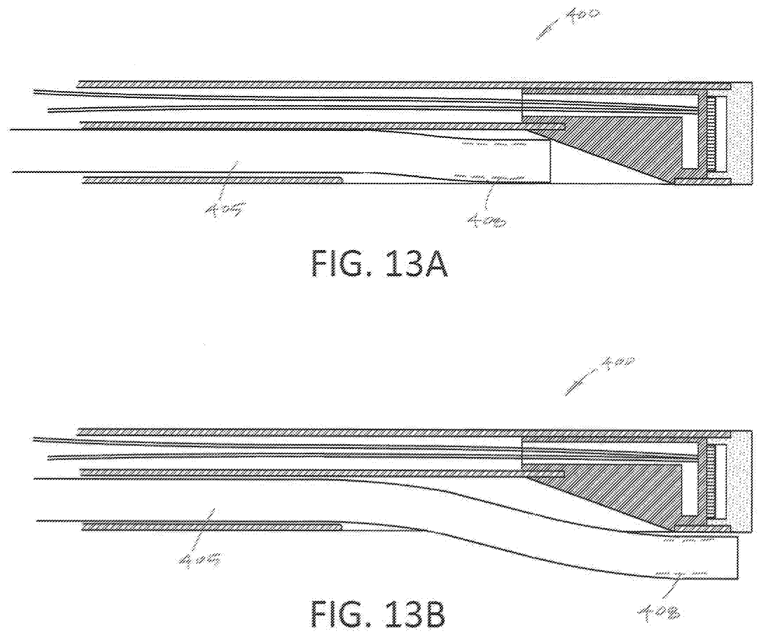

FIGS. 13A and 13B illustrate the distal portion of another endoscope 400 which is similar to the variation described previously. In this variation, an outer constraining sleeve is not required. The inner sleeve 405 which carries a working channel 408 again comprises a resilient material which can be moved axially between a non-extended position (FIG. 13A) and an extended position (FIG. 13B) to guide the flexible shaft of a tool. As can be seen in FIG. 13B, the inner sleeve 405 is resilient with multiple curves and is adapted to orient the working end of a tool as it exits the working channel 408 to be positioned in a substantially parallel path relative to the axis of the endoscope shaft. It can be understood that the inner sleeve 405 is keyed with the endoscope handle so the inner sleeve 405 cannot be rotated as it is moved axially.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A method for imaging and treating a patient's uterine cavity, comprising:
providing an endoscopic system comprising:
(a) a handle;
(b) a shaft having a rigid proximal shaft portion, an elastic distal portion, and at least two channels extending therethrough, wherein each channel has an open termination in the distal shaft portion and wherein the shaft is rotatably coupled to the handle;
(c) an image sensor carried by the distal shaft portion;
(d) at least one LED carried by the distal shaft portion;
(e) a control pad on the handle with control buttons for (i) the image sensor and (ii) the at least one LED; and
(f) a fluid management system configured to be coupled to said at least one channel in the shaft;
positioning a cervical seal in contact with the patient's cervical canal;
positioning the elastic distal portion of the shaft in the uterine cavity distal to the cervical seal;
imaging the uterine cavity with the image sensor;
coupling the at least one channel in the shaft to a fluid source in the fluid management system and at least another channel in the shaft to a negative pressure source in the fluid management system to circulate a fluid in the fluid management system through the uterine cavity;
advancing a straight tool through an entire length a working channel and through the distal shaft portion into the uterine cavity, wherein advancing the tool of the working channel contacts a surface of the distal shaft portion to deflect the elastic distal portion to allow advancement of the tool distally outward from the shaft;
controlling operation of (i) the image sensor and (ii) the at least one LED using the control panel;
rotating the shaft relative to the handle; and
using the tool to treat the patient's uterine cavity.

2. The method of claim 1, wherein advancing the straight tool through the working channel causes a central axis of the working channel in the distal portion to deflect relative to a central axis of the shaft.

3. The method of claim 1, wherein the tool remains straight as the distal portion deflects.

4. The method of claim 1, wherein the image sensor is an optical image sensor having a field-of-view projecting in a generally distal direction relative to the distal portion.

5. The method of claim 1, wherein the distal portion of the shaft comprises a shape-adjustable component.

6. The method of claim 1, wherein the at least one channel and the at least another channel comprise the working channel and a flow channel which extends from the handle to an open termination in the distal shaft portion.

7. A method as in claim 1, wherein positioning the elastic distal portion comprises advancing the elastic distal portion through the cervical seal into the uterine cavity.

8. A method as in claim 1, wherein the cervical seal is disposed over a sliding sleeve on the shaft.
